(19) United States
(12) Patent Application Publication
(10) Pub. No.: US 2007/0083100 A1
(43) Pub. Date: Apr. 12, 2007

(54) Ventriculostomy Catheter with In Situ Ultrasound Capability

(76) Inventor: Sebastian Schulz-Stubner, Coralville, IA (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/458,822

(22) Filed: Jul. 20, 2006

Related U.S. Application Data
(60) Provisional application No. 60/701,620, filed on Jul. 20, 2005.

(51) Int. Cl. A61B 5/05; A61B 8/14

(52) U.S. Cl. 600/407; 600/463; 128/916

(57) ABSTRACT

A ventriculostomy instrument comprises a ventriculostomy catheter having ultrasound capabilities. The ventriculostomy catheter can include a catheter wall defining a central lumen passageway, and a stylet device removably received in the central lumen passageway. An ultrasound probe is disposed in the catheter wall and remains in place at the distal end of the catheter when the stylet is removed. Thus it may be used to provide substantially continuous ultrasound imaging and monitoring during positioning, during fluid draining or during the insertion of medical instruments into the brain via the central lumen passageway of the catheter.

### RELATED APPLICATION DATA

This application claims the benefit of U.S. Provisional Application Ser. No. 60/701,620 filed Jul. 20, 2005, the disclosure of which is incorporated herein by reference.

### FIELD OF THE INVENTION

The present invention relates to ventriculostomy catheters and surgical systems utilizing the same. More particularly, but not exclusively, the present invention relates to a ventriculostomy catheter system with in situ ultrasound capability, wherein the ultrasonic element can remain in place at the working end of the catheter, independent of the removal of the stylet after the catheter has been positioned in its desired location.

### BACKGROUND OF THE INVENTION

Ventriculostomy catheters are commonly used for diagnostic and therapeutic purposes in a variety of patients with severe neurological injury, such as subarachnoid hemorrhage (SAH), traumatic brain injury (TBI), or stroke. Existing ventriculostomy catheters are capable of measuring intracranial pressure and draining cerebral spinal fluid (CSF). External ultrasound imaging via the use of transcranial or fontanelle windows can be unsatisfactory, for example due to limitations on frequency of use, patient discomfort, or variability of imaging quality depending on bone thickness and window quality.

Stylets are used to facilitate navigation and placement of catheters at desired locations, and then removed from the catheters once proper placement has been achieved, to allow for the use of other instruments within the catheters. U.S. Pat. No. 5,690,117, incorporated herein by reference, describes an ultrasound probe incorporated into the stylet to facilitate navigation and placement of the catheters. One drawback of this approach is that the ultrasound probe must be withdrawn from the catheter with the stylet to allow for access of other instrumentation. An improved ventriculostomy catheter would be beneficial wherein the ultrasound probe may remain in place, and thus available for continued use, upon removal of the stylet.

### SUMMARY

In one aspect, a surgical system for ventriculostomy is provided comprising a ventriculostomy catheter having an elongated length and a distal end, the catheter also defining at least a first lumen open to the distal end. A stylet device, which may include a steering cable and fiber optics, is removably received in the first lumen, and an ultrasound probe is operably associated with the distal end of the catheter such that the ultrasound probe can remain in place when the stylet device is withdrawn from the first lumen. In one refinement, the cathether defines at least a second lumen and the ultrasound probe is slidably received within the second lumen. In another refinement, the ultrasound probe is integral with the distal end of the catheter. In another refinement, the ultrasound probe is operable to provide substantially continuous ultrasound imaging during advancement of the ventriculostomy catheter or when the ventriculostomy catheter is positioned at a desired location in a ventricle of a patient's brain.

In another aspect, a method of use is provided comprising providing an ultrasound probe at the distal end of a vertriculostomy catheter, removably positioning a stylet in the working lumen of the catheter, guiding the stylet and catheter to a desired location in a brain, and after the guidance, removing the stylet while leaving the ultrasound probe in place at the distal end of the catheter. In one refinement, the guidance is under ultrasound visualization provided by the ultrasound probe. In another refinement, the guidance includes visualizing with fiber optics carried by the stylet. In another refinement, Doppler studies in the brain are performed with the ultrasound probe. In another refinement, the brain is visualized with the ultrasound probe after removal of the stylet. In another refinement, fluid is withdrawn and/or other instruments are inserted from the desired location.

In another aspect, a surgical system is provided comprising a steerable ventriculostomy catheter having an elongated length and a distal end, the catheter defining at least a first lumen open to the distal end, a steering stylet adapted to be removably received in the first lumen and to steer the distal end of the catheter to a desired location in a brain, and an ultrasound probe operably associated with the distal end of the catheter such that the ultrasound probe can remain in place when the steering stylet is withdrawn from the first lumen. In one refinement, the ultrasound probe includes one or more transducer elements embedded in the distal end of the catheter. In another refinement, the system includes an imaging device responsive to signals from the ultrasound probe for providing a visual display of the desired location in the brain.

In another aspect, a medical device comprises a ventriculostomy catheter having a proximal end, a distal end, and a lumen wall defining a central lumen passageway. The central lumen passageway is configured to selectively and slideably receive a stylet instrument. Additionally, the medical device comprises an ultrasound device in fixed engagement with the lumen wall of the ventriculostomy catheter to provide ultrasound imaging and monitoring.

In another aspect, a ventriculostomy instrument comprises a ventriculostomy catheter in engagement with an ultrasound probe. The ventriculostomy catheter has a distal end, a proximal end, and a catheter wall defining a central lumen passageway. The instrument further comprises a stylet device slideably received in the central lumen passageway. The ultrasound probe is disposed in the catheter wall of the ventriculostomy catheter to provide ultrasound imaging and monitoring.

In yet another aspect, a method comprises providing a ventriculostomy catheter having a proximal end, a distal end, a catheter wall defining a central lumen passageway, and an ultrasound probe positioned in the catheter wall. The method further comprises inserting a style device into the central lumen passageway, advancing the ventriculostomy catheter to a desired position in a brain ventricle and performing ultrasonic imaging and monitoring with the ultrasound probe at the desired position.

These and other aspects are discussed below.

### DESCRIPTION OF THE DRAWINGS

FIG. 1 is a view of a ventriculostomy catheter positioned in the brain.

FIG. 2 is a partial cross-sectional side view of a ventriculostomy catheter positioned in the brain.

FIG. 3 is a partial cross-sectional side view of a distal end portion of a ventriculostomy catheter and stylet.

FIG. 4A is a partial cross-sectional side view of a portion of the FIG. 3 ventriculostomy catheter with instrumentation replacing the stylet.

FIG. 4B is a partial perspective side view of the portion of the ventriculostomy catheter with instrumentation positioned therein as shown in FIG. 4A.

FIG. 5 is a partial cross-sectional side view of an alternative arrangement for the distal end portion of a ventriculostomy catheter and stylet.

### DESCRIPTION OF THE PREFERRED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein are contemplated as would normally occur to one skilled in the art to which the invention relates.

One embodiment of the present invention includes a rigid or flexible ventriculostomy catheter with one or more ultrasonic transducers operably associated at its distal end. The transducers are configured to remain in the distal end of the catheter even after another component (such as an insertion stylet) is removed. In one form, the transducers are embedded in the distal end, and in another form they are provided in lumens separate from the main lumen (e.g. stylet lumen) of the catheter. The transducers, coupled to appropriate electronics outside the patient, may be used to visualize placement of the catheter in the brain, to visualize placement of other instruments into the brain through the catheter, and/or to perform measurements on the brain (e.g. fluid flow rates and/or flow profiles). A separate stylet may be provided to guide the distal end of the catheter into position in the brain, for example under visualization provided by fiber optics in the stylet and/or under ultrasonic visualization provided by the transducers. The inventive catheter allows imaging from inside the brain and may be used to detect hydrocephalus, brain swelling, intracranial hemorrhage, and/or to measure intracerebral blood flow. Alternatively or in addition, the catheter may be used to aid in the diagnosis, treatment, and monitoring of patients with TBI, SAH and stroke.

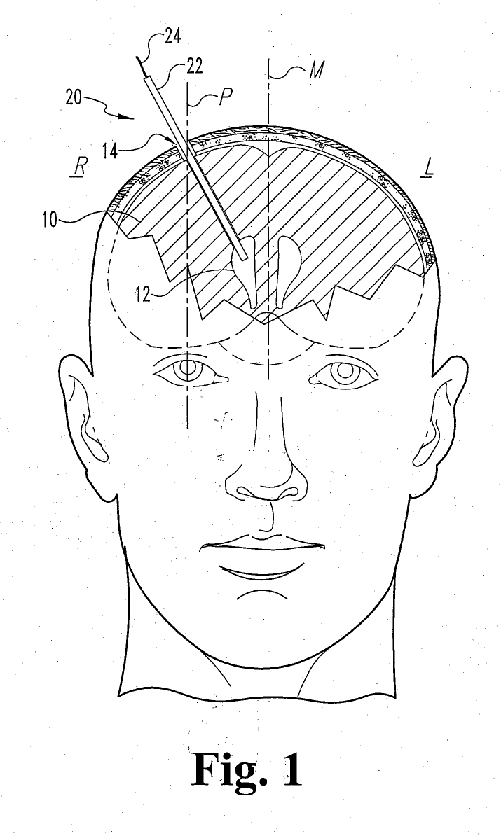

FIG. 1 illustrates a patient's brain 10 with a ventriculostomy catheter device 20 inserted therein. Brain 10 includes a right side R and a left side L, and includes a midline M and a pupil line P. In the illustrated embodiment, the device 20 is inserted through burr hole 14 and into right ventricle 12. Device 20 generally includes a catheter 22 with a cable 24 extending to operator monitoring equipment (not shown). The cable 24 may include a bundle of cables and is used to send and receive signals to one or more ultrasonic transducers at the distal end of catheter 22, as explained more fully below.

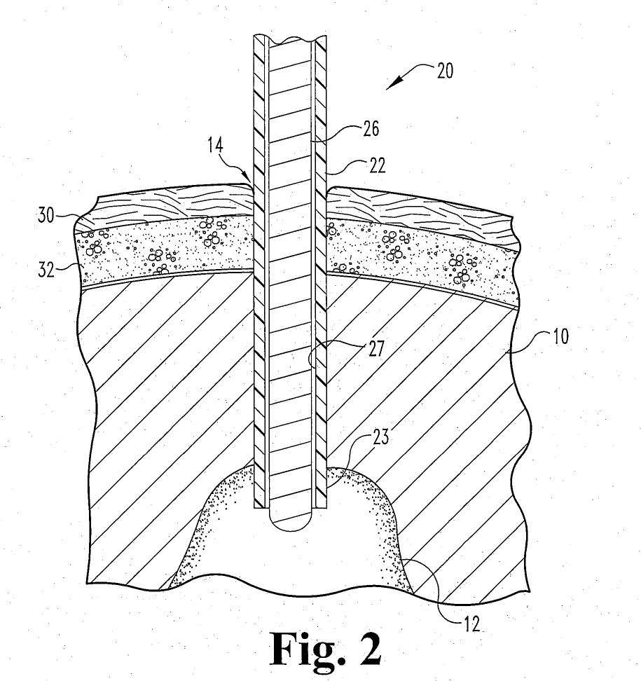

FIG. 2 is a cross-sectional view of device 20 inserted in brain 10. Device 20 is inserted through hole 14, through skin and muscle layer 30 and bone layer 32, into ventricle 12. Device 20 includes a stylet 26 to assist in guidance of the catheter 22 into position, for example with the distal end 23 of catheter 22 in fluid communication with ventricle 12. A steering cable (not shown) in stylet 26 is used to provide lateral guidance to stylet 26 and also catheter 22 as catheter 22 is longitudinally advanced. Proximal portions of stylet 26 and catheter 22 (not shown) are coupled or in engagement such that they are advanced longitudinally together. Alternatively or in addition, distal portions of stylet 26 and lumen 27 of catheter 22 are configured such that stylet 26 positively engages with catheter to provide or assist in longitudinal advancement thereof. As illustrated, the distal tip of stylet 26 is shown extending out the distal tip of catheter 22, but it is to be appreciated that stylet may be contained entirely within lumen 27 of catheter 22 during the advancement. Once catheter 22 has reached a desired location, stylet 26 may be removed to accommodate the insertion and use of various other instruments or fluid drainage through catheter 22.

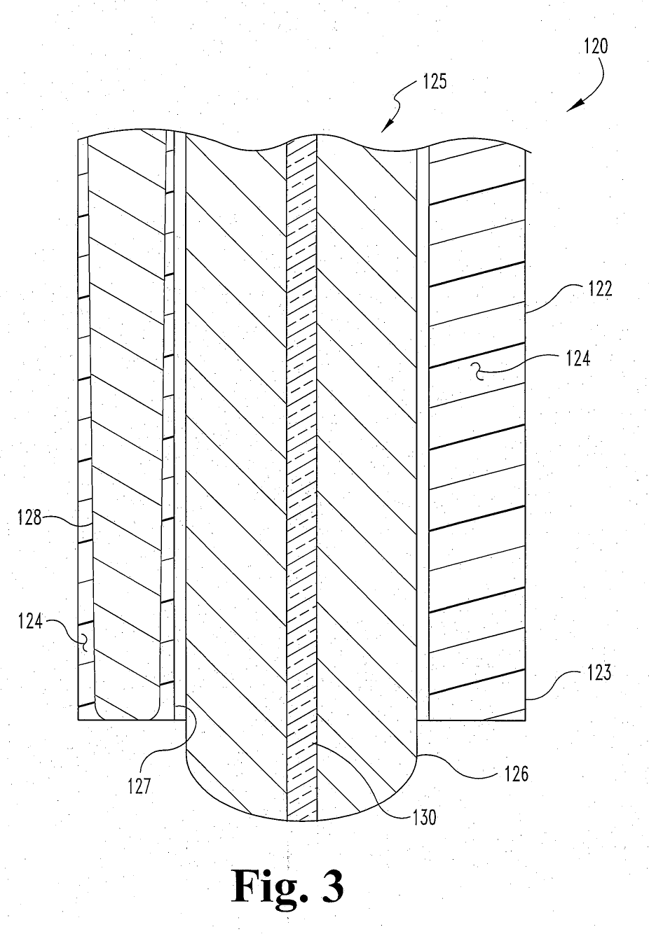

FIG. 3 illustrates a partial cross-sectional view of the distal end of a ventriculostomy catheter device 120 including a catheter 122 with lumen wall 124. Lumen wall 124 of catheter 122 defines a central lumen passageway 125 defined by interior surface 127. A stylet 126 is inserted into passageway 125. In the illustrated embodiment, stylet 126 carries fiber optics 130. Additionally, ultrasonic probe 128 is positioned within lumen wall 124 of catheter 122 and is connected via cable(s) 24 (FIG. 1) to a processing device and display (not shown) for producing 2-D ultrasound images. Fiber optics 130 can be used to assist in the navigation and placement of catheter 122 at a desired location. Alternatively, stylet 126 may be provided without fiber optics 130. Once at a desired position, stylet 126 is removed from catheter 122 to allow for insertion and use of other appropriate instruments or other functions, such as draining CSF for example. Ultrasonic probe 128, positioned in lumen wall 124 of catheter 122, remains in situ with catheter 122 to provide for continuous imaging and monitoring while catheter 122 is in place. After stylet 126 has been removed from catheter 122, instruments may be inserted through passageway 125 to access the brain under ultrasound supervision.

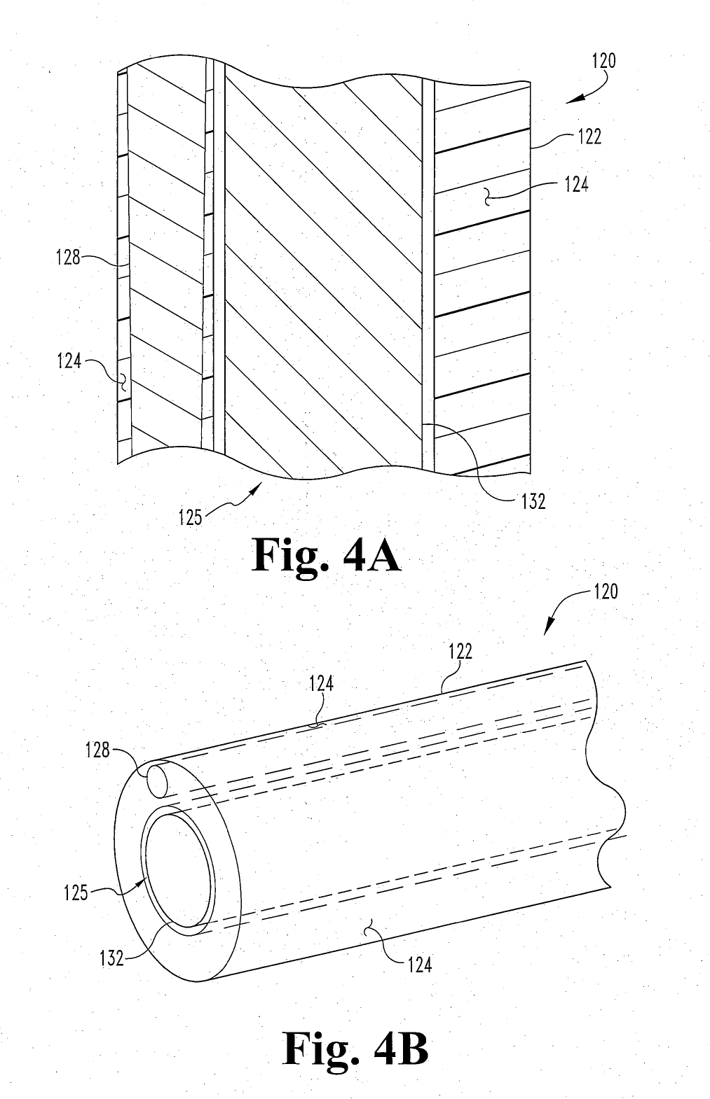

FIG. 4A illustrates a partial cross-sectional view of the FIG. 3 catheter with stylet 126 replaced with another instrument 132. As illustrated, catheter 122 has lumen wall 124 and defines central lumen passageway 125. Device 120 includes ultrasonic probe 128 disposed within lumen wall 124 of catheter 122 to permit continuous imaging and monitoring of brain areas relative to catheter 122. The positioning of ultrasonic probe 128 in lumen wall 124 allows for insertion and use of an instrument 132 inside passageway 125 of catheter 122, upon removal of stylet 126. It should be appreciated that instrument 132 can be any appropriate tool or instrument used in conjunction with a catheter as would generally occur to one skilled in the art.

FIG. 4B is a perspective view of ventriculostomy catheter device 120, illustrating the positioning of ultrasound probe 128 within wall 124. The configuration of the illustrated embodiment allows for the insertion of instrument 132 within passageway 125 of catheter 122, while maintaining imaging by ultrasonic probe 128 of the surrounding area. Ultrasonic probe 128 permits continuous imaging and monitoring of intracranial brain areas relative to catheter 122. It should be appreciated that ultrasonic probe 128 can be positioned in or onto catheter 122 in various manners as would generally occur to one skilled in the art. In certain embodiments, ultrasonic probe 128 is fixably positioned inside wall 124, for example by being embedded therein. In other embodiments, ultrasonic probe 128 is adjustably positioned in wall 124, for example by being provided in an ultrasound probe lumen defined therein, to allow for advancement or removal of ultrasonic probe 128 as desired.

A separate ultrasound probe lumen defined in the catheter wall 124 may be open at the distal end to allow at least a portion of the ultrasound probe to be extended out the distal end of catheter 122. Alternatively, an ultrasound probe lumen may be partially closed in such a manner that the lumen fluidly communicates with the external environment but the ultrasound probe cannot extend from the lumen. Alternatively, an ultrasound probe lumen may be a closed or blind lumen such that there is no fluid communication between the probe and the brain environment.

In one preferred embodiment, the ventriculostomy catheter is approximately 6 millimeters (mm) in diameter near the distal end, with the lumen wall being approximately 2 mm in thickness. However, it should be appreciated that the lumen wall can have a greater or lesser thickness to accommodate positioning of the ultrasonic probe therein. Additionally, in certain embodiments, the lumen wall can include a nonuniform thickness, for example having a thicker wall region where the ultrasonic probe is positioned. Further, it should be appreciated that the overall dimensions of the catheter can vary as would occur to one skilled in the art.

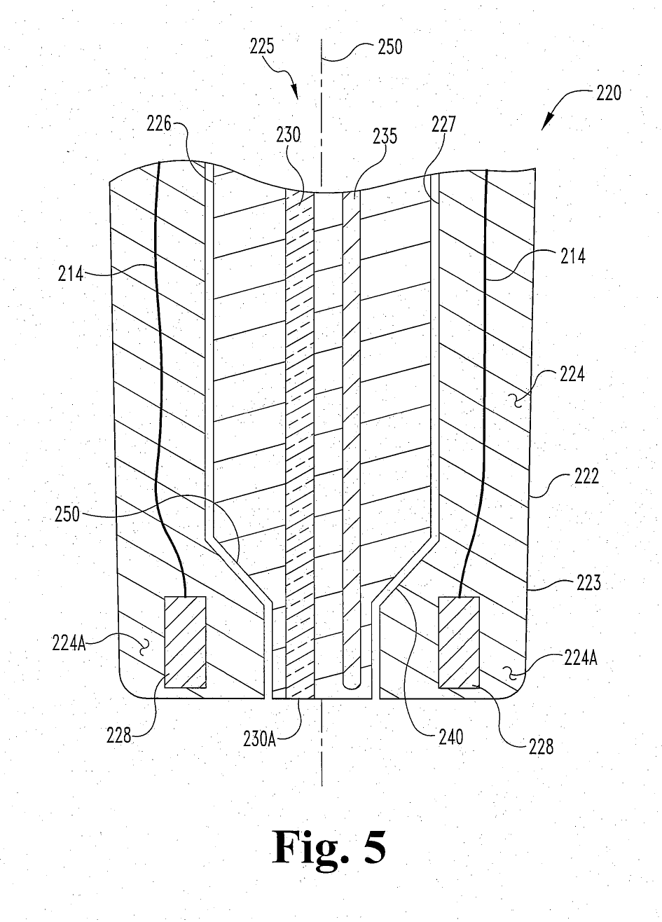

Turning now to FIG. 5, an alternative arrangement for the distal end portion of a ventriculostomy catheter and stylet is depicted. Device 220 includes a stylet 226 received in the generally centrally disposed lumen 225 of catheter 222. Stylet 226 includes a steering wire 235 operable to impart flexion orientation to stylet 226 and catheter 222. Stylet 226 also includes fiber optics 230 terminating in window 230a, which may be a lens, to allow direct visualization via the stylet optics.

The inner lumen 225 of catheter 222 has walls 227 defining a generally cylindrical cross section along most of the length of the device 220, but at the distal end 223 the inner dimension (e.g. diameter) of lumen 225 is reduced. As illustrated, the inner dimension of lumen 225 is reduced by virtue of inwardly disposed tapered section 240. The outer diameter of stylet 226 is shaped in correspondence with the inner surface 227 of lumen 225 and includes transition regions 250 that abut sections 240 of catheter 222 in a positive stop relationship, thereby preventing the distal tip of stylet 226 from extending beyond the distal tip of catheter 222. Longitudinal engagement of stylet 226 with distal end 223 of catheter 222 permits stylet 226 to be used to impart a longitudinally advancing force to catheter 222 and/or to more precisely steer the distal end 223 of catheter 222.

The distal end 223 of catheter 222 is tapered to facilitate smooth insertion, but the outer dimension of distal end 223 does not taper as abruptly as sections 240. As a result, the catheter wall 224 has thicker portions 224a on either side of the distal opening of lumen 227. One or more ultrasonic transducers 228 may be positioned in these thicker portions 224a with their respective signal lines 214 extending proximally in catheter wall 224.

Transducers 228 may be arranged in a variety of useful configurations to accomplish the objectives for the ultrasound probe described herein. For example, in one form, transducers 228 are an array of directional transducers oriented to send and receive acoustic signals along one or more transmission axes. Defined relative to the longitudinal axis 250 of the distal end 223 of catheter 222, such transmission axes may be within about 45° of parallel to axis 250, for example within about 25° or within about 15° of parallel thereto. Such transmission axes may also be transverse to axis 250. For example transmission axes of transducers 228 may be within about 45° of orthogonal to axis 250, for example within about 25° or within about 15° of orthogonal thereto. In these or in other forms, transducers 228 and/or their transmission axes may be spaced at different angular orientations about axis 250, for example generally equally angularly spaced.

Transducers 228 may be coupled to appropriate signal processors and output devices to accomplish the objectives for the ultrasound probe described herein. For example, in one form, a signal processor and an imaging device (such as a computer screen) is provided that is responsive to signals from the transducers to provide a substantially real time visual display. The display may be 2 dimensional or 3 dimensional and the display may be of local objects in the brain (e.g. tissue, blood or fluid flow) and/or the relative relationship between such objects and surgical instruments (such as the catheter or objects inserted there through).

While the invention has been illustrated and described in detail in the drawings and foregoing description, the same is to be considered as illustrative and not restrictive in character. Only certain embodiments have been shown and described, and all changes, equivalents, and modifications that come within the spirit of the invention described herein are desired to be protected. Any theory, mechanism of operation, proof, or finding stated herein is meant to further enhance understanding of the present invention and is not intended to limit the present invention in any way to such theory, mechanism of operation, proof, or finding. Thus, the specifics of this description and the attached drawings should not be interpreted to limit the scope of this invention to the specifics thereof. It is to be understood that when words such as “a”, “an”, and “at least one” are used, there is no intention to limit to only one item unless specifically stated to the contrary. Furthermore, when the language “at least a portion” and/or “a portion” is used, a portion and/or the entire item may be present. Finally, all publications, patents, and patent applications cited in this specification are herein incorporated by reference to the extent not inconsistent with the present disclosure as if each were specifically and individually indicated to be incorporated by reference and set forth in its entirety herein.

What is claimed is:

1. A surgical system, comprising:
a ventriculostomy catheter having an elongated length and a distal end, said catheter defining at least a first lumen open through the distal end;
a stylet device removably received in said first lumen; and
an ultrasound probe operably associated with the distal end of said catheter such that said ultrasound probe remains in place at the distal end of said catheter when said stylet device is withdrawn from said first lumen.

2. The system of claim 1 wherein said cathether defines at least a second lumen and said ultrasound probe is disposed within said second lumen.

3. The system of claim 2 wherein said second lumen is closed at the distal end of said catheter.

4. The system of claim 1 wherein said ultrasound probe is integral with the distal end of said catheter.

5. The system of claim 3 wherein said stylet device includes fiber optics and a steering cable.

6. The system of claim 5 wherein said ultrasound probe is operable to provide substantially continuous ultrasound imaging during advancement of said ventriculostomy catheter.

7. The system of claim 6 when said ultrasound probe is operable to provide substantially continuous ultrasound imaging when said ventriculostomy catheter is positioned at a desired location in a ventricle of a patient's brain.

8. The system of claim 5 when said ultrasound probe is operable to provide substantially continuous ultrasound imaging when said ventriculostomy catheter is positioned at a desired location in a ventricle of a patient's brain.

9. A method, comprising:
(a) providing an ultrasound probe at the distal end of a ventriculostomy catheter;
(b) removably positioning a stylet in a main lumen of the catheter;
(c) guiding the catheter to a desired location in a brain; and
(d) after said guiding (c), removing the stylet while leaving the ultrasound probe in place at the distal end of said catheter.

10. The method of claim 9 wherein said guiding (c) includes visualizing with the ultrasound probe.

11. The method of claim 10 wherein said guiding (c) includes visualizing with fiber optics carried by the stylet.

12. The method of claim 9 further comprising:
(e) performing Doppler measurements in the brain with the ultrasound probe.

13. The method of claim 9 further comprising:
(e) after said removing (d), visualizing at least a portion of the desired location with the ultrasound probe.

14. The method of claim 13 wherein the desired location is in a ventricle, the method further comprising:
(f) withdrawing fluid from the ventricle.

15. The method of claim 14 wherein said withdrawing (f) is with a fluid draining instrument inserted into the main lumen.

16. The method of claim 13 wherein (e) includes visualizing the position of a device in the main lumen.

17. The method of claim 9 further comprising:
(e) withdrawing fluid from the desired location while visualizing with the ultrasound probe.

18. A surgical system comprising:
a steerable ventriculostomy catheter having an elongated length and a distal end, said catheter defining at least a first lumen open through the distal end;
a steering stylet adapted to be removably received in said first lumen and to steer the distal end of said catheter to a desired location in a brain; and
an ultrasound probe operably associated with the distal end of said catheter such that said ultrasound probe remains in place at the distal end of said catheter when said steering stylet is withdrawn from said first lumen.

19. The system of claim 18 wherein said ultrasound probe includes one or more transducer elements embedded in the distal end of said catheter.

20. The system of claim 18 further comprising an imaging device responsive to signals from said ultrasound probe for providing a visual display of the desired location in the brain.
